(19) United States
(12) Patent Application Publication
(10) Pub. No.: US 2008/0023346 A1
(43) Pub. Date: Jan. 31, 2008

(54) Protected Stent Delivery System and Packaging

(75) Inventor: Carlos Vonderwalde, Richmond (CA)

Correspondence Address: Martin D. Moynihan;PRTSI, Inc., P.O.Box 16446, Arlington, VA, 22215, US

(73) Assignee: Design & Performance - Cyprus Limited, Nicosia (CY)

(21) Appl. No.: 11/629,184

(22) PCT Filed: Jun. 9, 2005
(86) PCT No.: PCT/IL05/00615

§ 371 Date: Dec. 11, 2006

(30) Foreign Application Priority Data
Jun. 10, 2004 (US) 10/865379

(51) Int. Cl. B65D 81/22; A61F 2/82; A61F 2/84; A61L 2/20

(52) U.S. Cl. 206/210; 422/28; 623/1.11; 623/1.42

(57) ABSTRACT

The present invention is directed to sterile packaging for medical devices or components where the medical device or component (e.g., a stent) is protected, at least in part with a sheath or the like configured to maintain a protective fluid about a portion of the device or component. The sheath preferably has one or more ports to add fluid to or withdraw fluid from the interior of the protective sheath.

### BACKGROUND OF THE INVENTION

The present invention relates generally to sterilized medical devices and packaging for sterilized medical devices. In particular, the present invention relates to the packaging of sterilized environmentally sensitive medical devices, such as stents including components made of biological materials or including active pharmaceutical ingredients. The present invention also relates to surgery, and in particular to a stent delivery device.

Stents are usually cylindrically devices, often of metal, configured to be implanted into a patients body lumen to maintain the patency thereof. Stents are particularly useful in the treatment and repair of blood vessels after or during percutaneous transluminal coronary angioplasty (PTCA), percutaneous transluminal angioplasty (PTA), or atherectomy and reduce the possibility of restenosis. Stents are known to be used in treating vulnerable plaque. Stents are usually delivered in a radially compressed condition to a required location and then deployed at that location by expansion to support the vessel and thereby vessel patency. Stents are also used to support and hold back a dissected arterial lining after an angioplasty procedure to avoid occlusion of the arterial passageway.

Many stent designs are known. One of the difficulties encountered in prior art stents involve maintaining the radial rigidity needed to hold open a body lumen, while at the same time maintaining the longitudinal flexibility of the stent to facilitate delivery through and deployment at the often tortuous path of bodily lumen.

PTCA is a well-established minimally invasive procedure for the treatment of heart disease, wherein a balloon catheter is advanced within the patient's vasculature until the balloon on the catheter is disposed within the arterial blockage and the balloon is inflated to expand the blockage to thereby increase the blood flow therethrough. In a typical PTCA procedure, a guiding catheter is first percutaneously inserted into the patient's cardiovascular system either through the brachial or the femoral arteries, and is advanced until the distal tip of the guiding catheter is seated within the ostium of the desired coronary artery. A balloon dilatation catheter is then advanced out of the guiding catheter into a patient's coronary artery through the inner lumen of the guiding catheter, until the balloon at the distal portion of the catheter is disposed within the desired region of the patient's artery. The balloon is inflated and deflated one or more times as required to re-open the arterial passageway and thereby permit blood flow volume to increase once the catheter is removed.

Most angioplasty procedures today involve placement of a stent at the site to minimize restenosis and generally provide scaffolding support to the arterial region. Typically, a stent is delivered with the balloon expansion to dilate the stenosis. However, a stent may be delivered after the angioplasty procedure is completed with another balloon catheter which is similar to the balloon catheter used for the angioplasty procedure.

There are generally two types of catheters used in PTCA/stent delivery procedures, namely the rapid exchange type balloon catheters and the less common over-the-wire type balloon catheters.

A rapid exchange type balloon catheter has a relatively short guide wire receiving lumen extending through a distal portion of the catheter with one guide wire port at the distal end of the catheter and another guide wire port spaced about 5 to about 50, usually about 10 to about 40 cm, from the distal end. Such catheters allow for the rapid exchange of the catheter without the need for an exchange wire or adding a guide wire extension to an in-place guide wire. Over-the-wire balloon catheters have guide wire lumens which extend the entire length of the catheter and require guide wire extensions or exchange wires to exchange the catheter.

Key features for effective stent delivery include delivery of the stent through tortuous bodily lumen without damage to the stent or displacement of the stent from the balloon. This requires that the catheter be highly responsive to the controlled advancement of the balloon catheter with optimal pushability from the proximal shaft section, and at the same time, retaining overall optimal flexibility for advancement.

Many types of medical devices must be sterile. Further, most such medical devices must be delivered in some sort of sealed sterile packaging, e.g., pouch, bag, tube, box, or other container, to maintain sterility during storage and delivery as well as to protect the device from physical damage.

It is known to sterilize the packaging and the device separately and only subsequently to pack the device in the packaging under sterile conditions. It is preferred, however, to first pack the device in the packaging, seal the packaging and subsequently to sterilize the device and the packaging concurrently. A preferred type of medical device packaging is made, at least in part, of spun high-density polyethylene (HDPE) (e.g., Tyvek®, which is available from Medical Packaging Division of E. I DuPont de Nemours and Company) which is strong, puncture-resistant and tear-resistant. Spun high-density polyethylene is transparent to e-beams and gamma rays used in irradiative sterilization of medical devices. Importantly, spun high-density polyethylene is permeable to air and common sterilizing gases (e.g., ethylene oxide) but constitutes an impassible barrier to moisture, bacteria, viruses, and other substances which may compromise the sterility or are otherwise detrimental to the device and the packaging.

While spun high-density polyethylene packaging is widely used for many medical devices such as catheters, stents, surgical instruments, and probes, spun high-density polyethylene is inadequate as a packaging material for medical devices including components which are subject to oxygen degradation or which must be maintained under particular s. For example, in U.S. Pat. No. 6,699,277 a stent is described having a jacket formed of heterologous tissue such as pericardial tissue, e.g., bovine, porcine or equine pericardial tissue. If not stored under exact conditions, or if exposed to oxygen or sterilizing gases, such a jacket may degrade. Similarly, drug-eluting stents (DES) are coated with a plastic coating incorporating an active pharmaceutical ingredient (API) (e.g., paclitaxel or rapamycin). Drug eluting stents are made at a central fabrication facility and must be distributed to the end user in sterile packaging. The API incorporated in a drug-eluting stent may be adversely affected over the time required for distribution and storage after exposure to ethylene oxide or oxygen that permeate through spun high-density polyethylene. One problem known with jacketed stents or with drug eluting stents is that of transport and storage. It is preferred that such stents be produced under carefully controlled and repeatable conditions at a central, easy to regulate, location. Subsequent to production, the stents must be transported and stored until such time as the stent is used. The jackets of some jacketed stents are made of biological tissue that may be damaged by common sterilizing methods and must be stored under carefully controlled conditions, e.g., in a specific solution and protected from oxygen. Similarly, drug-eluting stents must also be stored and transported under careful conditions to ensure that there is no change in the quality of the active pharmaceutical ingredient or in the drug-eluting matrix.

Thus, there is a need for a package for medical devices devoid of the shortcomings of the medical device packages known in the art.

### SUMMARY OF THE INVENTION

The present invention successfully addresses at least some of the shortcomings of prior art by providing an improved package for medical devices or components, such as: jacketed stents having a decomposable or degradable jacket; drug eluting stents, where the stent coating or drug incorporated into the stent coating is easily degradable; and. The present invention is also useful with pre-crimped covered or coated stents which are not compatible with standard sterilization procedures and which need to be protected and/or kept moist.

The protected delivery system embodying features of the invention preferably includes a delivery catheter which has an elongated shaft, a distal extremity on the elongated shaft, and a stent expanding member on the distal extremity of the catheter. A jacketed or coated stent is mounted on the stent expanding member on the distal extremity of the catheter. A sealable container or sheath is disposed about the distal extremity of the catheter having an interior receiving the expandable member and the stent mounted thereon. The sheath or container may then be sealed. The container or sheath is configured to maintain a body of protective fluid about the mounted stent to ensure that the device is ready to be used by the physician when the package is open with little or no preparation. The protective fluid may be a liquid, a slurry, a gel or a gas. The sheath is preferably provided with one or more sealable ports to add or withdraw fluid from the interior of the sheath. For example, the protective fluid may be withdrawn and the distal portion of the catheter and the mounted stent with a jacket or coating may be rinsed with sterile water or saline prior to introduction into the patient. The distal extremity of the catheter and the mounted stent are sterilized prior to placement of the protective fluid within the container or sheath and the sealing thereof.

The type of protective fluid maintained within the interior of the sheath will depend upon the protection needed or desired. For example, in the case of a stent with a jacket formed of pericardial tissue, the protective fluid might be glutaraldehyde. In other cases, the fluid may be sterile water or sterile saline. Gaseous fluids such as inert or otherwise non-reactive (to the coating or jacket or any drugs contained therein) gas such as Nitrogen may also be employed. The protective fluid may also contain a drug or diagnostic agent in order to impregnate the stent or the stent cover or to maintain a proper drug concentration.

The protected delivery system may be stored or transported as is and further sterilized on site. However, the protected delivery system is preferably part of a sterilized packaging system. For example, the packaging system may include a sealable container configured to receive the protected delivery system. The container may be formed at least in part of a material, e.g. a spun high-density polyethylene such as Tyvec® that is permeable to sterilizing gas such as ethylene oxide. The ethylene oxide may be mixed with up to 80% of otherwise non reactive gases. Other gaseous sterilizing agents include Aprolene and hydrogen peroxide.

In one packaging system embodying features of the invention, the container is a tray sealed with a cover which is configured to receive the protected delivery system. The sealed covered tray is formed in part of a material as described above which is permeable to the sterilizing gas but which impermeable to bacteria, viruses, water and other detrimental materials. The contents of the covered tray, the protected delivery system, may be sterilized by maintaining a sterilizing gas in contact with at least the portion of the covered tray which is formed of the permeable material. In this embodiment, the tray and cover are preferably sealed prior to sterilization.

In another packaging system embodying features of the invention, an unsealed covered tray is placed in a pouch or envelope that is formed at least in part of a material which is permeable to sterilizing gas and the pouch or envelope then sealed. The pouch or envelope is then subjected to a sterilizing gas atmosphere as described above to sterilize the covered tray and its contents.

A suitable material which is permeable to sterilizing gas and which generally has the requisite mechanical and other properties for sterile packaging for medical products is a spun high density polyethylene such as Tyvek® (2FS, 1059B and 10738).

The catheter utilized in the protected stent delivery system may be a conventional stent delivery catheter (over-the-wire or rapid exchange) or may be of the design described in co-pending application Ser. No. 10/735,548, filed on Dec. 12, 2003, which has been assigned to the present assignee. The delivery catheter for percutaneous deployment to the desired intra corporeal location generally has an elongated shaft with an inflation lumen and a guide wire lumen extending therein and an inflatable member or balloon which is disposed about a distal shaft section. An inflatable member is configured to expand a stent mounted on the exterior thereof.

The stent is mounted on the working section of the balloon for delivery, usually by crimping, and is generally the same length or a little shorter than the working length of the balloon. See for example U.S. Pat. No. 6,605,107.

The interior of the covered tray may be evacuated to aspirate sterilizing gas surrounding the pouch or covered tray to facilitate the passage of sterilizing gas through the permeable portion of the covered tray or pouch. The pressure of the sterilizing gas is maintained within the container interior at suitable levels for effective sterilization. The dwell time of the sterilizing gas within the container interior depends upon the nature and pressure of the sterilizing agent. After sterilization, the interior can be partially or totally evacuated of oxygen or an oxygen containing gas and replaced totally or partially with a non-reactive gas such as nitrogen. The interior may be flushed or purged with a non reactive gas one or more times in order to reduce the level of sterilizing gas within the interior.

In some cases, it may be further desirable to provide desiccant materials within the sealed covered tray or within the pouch in order to sequester any moisture which may remain within the pouch after final sealing. Alternatively, a small canister of the desiccant material may be provided within the enclosure, similar to the desiccant placed in conventional pharmaceutical packaging.

The present invention also provides a stent delivery device configured to maintain a stent in a ready to use state and to prevent sensitive components of a stent from degrading. According to the teachings of the present invention there is provided a stent delivery device for a stent, comprising: a an elongated shaft that is a component of a stent delivery catheter (or substantially an entire stent delivery catheter); b. a stent delivery member on a distal extremity of the elongated shaft; c. a stent mounted on the stent delivery member; and d. a container configured to maintain a fluid within an interior volume, holding the stent delivery member and the stent within the interior volume.

In an embodiment of the present invention the stent is a self-expanding stent and the stent delivery member is the member which carries the stent to the deployment location and, in some embodiments, allows or causes the stent to expand.

In embodiments of the present invention the stent is a non-self expanding stent and the stent delivery member is a stent expanding member (e.g., an inflatable member such as a stent expanding balloon).

As noted above, in embodiments of the present invention at least part of the container sealingly engages the elongated shaft that passes through the part of the container.

As noted above, in embodiments of the present invention the stent delivery device further comprises at least one port (and even at least two) configured to allow fluid communication to the interior volume. Such a port is preferably sealable and even more preferably reversibly sealable. Sealable and reversibly sealable ports can be implemented using, for example, such components as mechanical valves, diaphragms, self sealing diaphragms, ports provided with caps, covers and plugs.

As noted above, in embodiments of the present invention the container is substantially a sheath (e.g., a tubular sheath) having a distal end, a proximal end and an inner lumen in which the stent is held. In embodiments of the present invention, the proximal end of the sheath sealingly engages the elongated shaft. In embodiments of the present invention, the sheath includes at least one (or even at least two) port configured to allow fluid communication with the interior volume. In embodiments, a port is found at the distal end (especially the tip) of the sheath and/or at the side of the sheath.

As noted above, in embodiments of the present invention the container is at least partially filled with a fluid as discussed above. In an embodiment of the present invention, the fluid in the container is a glutaraldehyde solution. Such a solution is exceptionally useful when the stent has an inner or an outer jacket, especially when the jacket is of a biological tissue such as described in U.S. Pat. No. 6,468,300. In an embodiment of the present invention, the fluid in the container comprises an active pharmaceutical ingredient (API). Such a fluid is exceptionally useful when the stent includes an API, such as a drug eluting stent, e.g., drug eluting stent with a polymeric coating having an eluting drug incorporated therein, or a jacket of biological tissue or artificial tissue with an eluting drug incorporated therein. Such APIs include therapeutic agents, diagnostic agent, rapamycin and paclitaxel.

In embodiments of the present invention, the container is opaque to sterilizing radiation such as ultraviolet and/or gamma rays and/or e-beams.

In embodiments of the present invention, the container is impermeable to oxygen and/or sterilizing gases such as ethylene oxide.

The present invention also provides a stent in a package that is configured to maintain a stent in a ready to use state, to prevent sensitive components of the stent from degrading and also allows sterilization of the stent without damaging the stent components during the sterilization process. According to the teachings of the present invention there is also provided a packaged stent, comprising: a. a package including an interior volume configured to receive a stent delivery device as described above and to maintain sterility of the interior volume; and b. a stent delivery device as described above packaged within the interior volume of the package. In an embodiment, the package comprises: i. a support tray (configured to receive and/or support and/or hold at least part of the delivery catheter and/or container) and ii. a tray cover engaging the support tray so as to define the interior volume therebetween. In embodiments of the present invention, the package comprises a pouch (e.g., bag, sack, envelope) defining the interior volume, preferably also comprising a support tray (configured to receive and/or support and/or hold at least part of the delivery catheter and/or container).

As noted above, in embodiments of the present invention, the container of the stent delivery device is substantially entirely disposed within the interior volume of the package.

As noted above, in embodiments of the present invention at least part of the container of the stent delivery device is not disposed within the interior volume.

As noted above, in embodiments of the present invention the container of the stent delivery device is provided with at least one port (or at least two ports) configured to allow fluid communication with the interior volume wherein the port is accessible from a location outside the interior volume of the package without comprising sterile conditions of the interior volume of the package.

In embodiments of the present invention at least part of the package (e.g., the package in its entirety, a tray, a tray cover) is substantially transparent to sterilizing radiation, sterilizing radiation such as ultraviolet and/or gamma rays and/or e-beams.

As noted above, in embodiments of the present invention at least part of the package (e.g., the package in its entirety, a tray, a tray cover) is permeable to a sterilizing gas. Parts of the package can be rendered permeable to a sterilizing gas such as ethylene oxide if made, for example, from spun polyolefins such as high-density poly-ethylene.

As noted above, in embodiments of the present invention packaged stent, further comprises an oxygen scavenger in fluid communication with the interior volume of the package to ensure that oxygen remaining in the interior volume is neutralized

The present invention more generally provides a medical device in a package that is configured to prevent sensitive components of the medical device from degrading and also allows sterilization of the medical device without damaging any component during the sterilization process.

According to the teachings of the present invention there is also provided a packaged medical device, comprising: a. a package including an interior volume configured to receive a medical device and to maintain sterility of the interior volume and b. a medical device packaged within the interior volume of the package wherein a part of the medical device is held within a container configured to maintain a fluid within an interior volume of the container.

In embodiments of the present invention the medical device is a stent delivery device as described above. As noted above, in embodiments of the present invention part of the medical device held within the container includes biological tissue.

As noted above, in embodiments of the present invention the interior volume is at least partially filled with a fluid. As noted above, in embodiments of the present invention the fluid comprises an active pharmaceutical ingredient.

As noted above, in embodiments of the present invention the container is opaque to sterilizing radiation.

In an embodiment, the package comprises: i. a support tray (configured to receive and/or support and/or hold at least part of the delivery catheter and/or container) and ii. a tray cover engaging the support tray so as to define the interior volume therebetween. In embodiments of the present invention, the package comprises a pouch (e.g., bag, sack, envelope) defining the interior volume, preferably also comprising a support tray (configured to receive and/or support and/or hold at least part of the delivery catheter and/or container).

As noted above, in embodiments of the present invention, the container of the medical device is substantially entirely disposed within the interior volume of the package.

As noted above, in embodiments of the present invention at least part of the container of the medical device is not disposed within the interior volume.

As noted above, in embodiments of the present invention the container of the medical device is provided with at least one port (or at least two ports) configured to allow fluid communication with the interior volume wherein the port is accessible from a location outside the interior volume of the package without comprising sterile conditions of the interior volume of the package.

In embodiments of the present invention at least part of the package (e.g., the package in its entirety, a tray, a tray cover) is substantially transparent to sterilizing radiation, sterilizing radiation such as ultraviolet and/or gamma rays and/or e-beams.

As noted above, in embodiments of the present invention at least part of the package (e.g., the package in its entirety, a tray, a tray cover) is permeable to a sterilizing gas. Parts of the package can be rendered permeable to a sterilizing gas such as ethylene oxide if made, for example, from spun polyolefins such as high-density polyethylene.

In a preferred embodiment, the tray is, for example, of a rigid material and includes features to hold the medical device (see tray depicted in FIG. 5B) while the tray cover is a sheet of spun high-density polyethylene (e.g., Tyvec®) sealingly attached to the tray in the usual way.

As noted above, in embodiments of the present invention packaged stent, further comprises an oxygen scavenger in fluid communication with the interior volume of the package to ensure that oxygen remaining in the interior volume is neutralized

The present invention provides a method for packaging a medical device that includes sterilization of the device without damaging any component during the sterilization process.

According to the teachings of the present invention, there is also provided a method for packaging a medical device, comprising: a providing a medical device having a first part and a second part; b. placing the first part within a container configured to maintain a fluid within an interior volume of the container and sealing the container; c. packaging the medical device in an interior volume of a package configured to maintain sterility of the interior volume (and if necessary sealing so that if the interior volume was sterile, the sterility would be maintained); d. while the medical device is packed within the package, sterilizing at least part of the medical device.

In embodiments of the present invention, during (d) at least the second part of the medical device is sterilized.

In embodiments of the present invention, during (d) both the second part and the first part of the medical device are sterilized. In embodiments, during (d) also fluid in the interior volume is sterilized.

In embodiments of the present invention, fluid is added to the container. Preferably, the container is provided with sealable ports allowing access to the internal volume of the container, especially reversibly sealable ports. In embodiments where the ports emerge out from the package, fluid is added to the container before, during or after (c) and/or (d).

In embodiments of the present invention, before the container is sealed in (b), the first part is sterilized either before, during or after placing the first part in the container. For example, a first part (e.g., a jacketed stent is sterilized and then placed in the container; a jacketed stent is sterilized when being placed in the container by contact with a sterilizing fluid already contained in the container; a jacketed stent is placed in the container and then sterilized by the addition of a sterilizing fluid).

In embodiments of the present invention at least part of the package is permeable to a sterilizing gas, and the sterilizing at least part of the medical device in (d) includes exposing the package to a sterilizing gas so that the sterilizing gas penetrates into the interior volume of the package so as to sterilize the second part of the medical device. In embodiments of the present invention the container is impermeable to the sterilizing gas. For example, a stent delivery device of the present invention including a drug-eluting stent including an API impregnated jacket of bovine pericardium is to be packed in package which is permeable to ethylene oxide. Sterilization of the stent delivery device (excepting the components held in the container) are sterilized by exposure to ethylene oxide permeating through the package. The components held in the container (e.g., stent, stent jacket) are sterilized separately, for example before being placed in the container or alternatively during or after being placed in the container, for example by the addition of a sterilizing fluid to the container.

In embodiments of the present invention at least part of the package is substantially transparent to a sterilizing radiation, and the sterilizing at least part of the medical device includes irradiating the second part of the medical device through the substantially transparent part of the package so as to sterilize the second part. In embodiments of the present invention the container is opaque to the sterilizing radiation and thus the second part is shielded from the sterilizing radiation. In embodiments of the present invention at least part of the container is substantially transparent to the sterilizing radiation and thus the contents of the container (the first part and fluid if present) are exposed to and sterilized by the sterilizing radiation (e.g., a jacketed stent of a stent delivery device of the present invention is placed in the container together with a preservative fluid, e.g., saline or glutaraldehyde solution, neither the stent nor the fluid necessarily being sterile. The container is sealed and the stent delivery device packaged according to the teachings of the present invention where parts of both the package and the container are substantially transparent to sterilizing radiation. Irradiation with sterilizing radiation then sterilizes the elongated shaft (and associated stent delivery catheter if present) the stent, the stent jacket and the preservative fluid which have all been packed in non-sterile conditions.

The invention provides a simple, inexpensive protection and packaging of medical devices and components which are formed of or are coated or covered with degradable or labile materials.

These and other advantages of the invention will become more apparent from the following detailed description of the invention when taken in conjunction with the accompanying exemplary drawings.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is an elevational view of a protected stent delivery system, partially in section, which embodies features of the invention;

FIG. 2A is an exploded perspective view of a tray, the protected stent delivery system shown in FIG. 1 and a protective cover;

FIG. 2B is a partially exploded perspective view with the protected stent delivery system disposed within the recess of the tray and the cover;

FIG. 2C is a perspective view of the tray, the protected stent delivery system and the cover completely closed;

FIG. 2D is a perspective view of the closed covered tray being inserted into a sterilizable pouch;

FIG. 2E is an elevational view, in section of the covered tray in the sealed pouch.

FIG. 3 is a transverse cross sectional view of the covered tray shown in FIG. 2c, taken along lines 3-3;

FIG. 4 is a transverse cross sectional view of the covered tray shown in FIG. 2c, taken along lines 4-4;

FIG. 5A is an exploded, perspective view of a covered stent delivery system and packaging therefore;

FIG. 5B is an exploded, perspective view of a covered stent delivery system and packaging therefore shown in FIG. 5a with the stent delivery system disposed within the recess of the packaging tray; and

FIG. 5C is a perspective view of a covered stent delivery system (in phantom) disposed within packaging therefore.

### DETAILED DESCRIPTION OF EMBODIMENTS OF THE INVENTION

The present invention is of a stent delivery device, a packaged medical device (in particular a stent) and a method for packaging a medical device. The principles, uses and implementations of the teachings of the present invention may be better understood with reference to the accompanying description and figures. Upon perusal of the description and figures present herein, one skilled in the art is able to implement the teachings of the present invention without undue effort or experimentation. In the figures, like reference numerals refer to like parts throughout.

Before explaining at least one embodiment of the invention in detail, it is to be understood that the invention is not limited in its application to the details set forth herein. The invention can be implemented with other embodiments and can be practiced or carried out in various ways. It is also understood that the phraseology and terminology employed herein is for descriptive purpose and should not be regarded as limiting.

Generally, the nomenclature used herein and the laboratory procedures utilized in the present invention include techniques from the fields of medicine, biology, chemistry and engineering. Such techniques are thoroughly explained in the literature. Unless otherwise defined, all technical and scientific terms used herein have the same meaning as commonly understood by one of ordinary skill in the art to which the invention belongs. In addition, the descriptions, materials, methods and examples are illustrative only and not intended to be limiting. Methods and materials similar or equivalent to those described herein can be used in the practice or testing of the present invention. All publications, patent applications, patents and other references mentioned are incorporated by reference in their entirety as if fully set forth herein. In case of conflict, the specification herein, including definitions, will control.

As used herein, the terms “comprising” and “including” or grammatical variants thereof are to be taken as specifying the stated features, integers, steps or components but do not preclude the addition of one or more additional features, integers, steps, components or groups thereof. This term encompasses the terms “consisting of” and “consisting essentially of”.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

The term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Implementation of the methods of the present invention involves performing or completing selected tasks or steps manually, automatically, or a combination thereof.

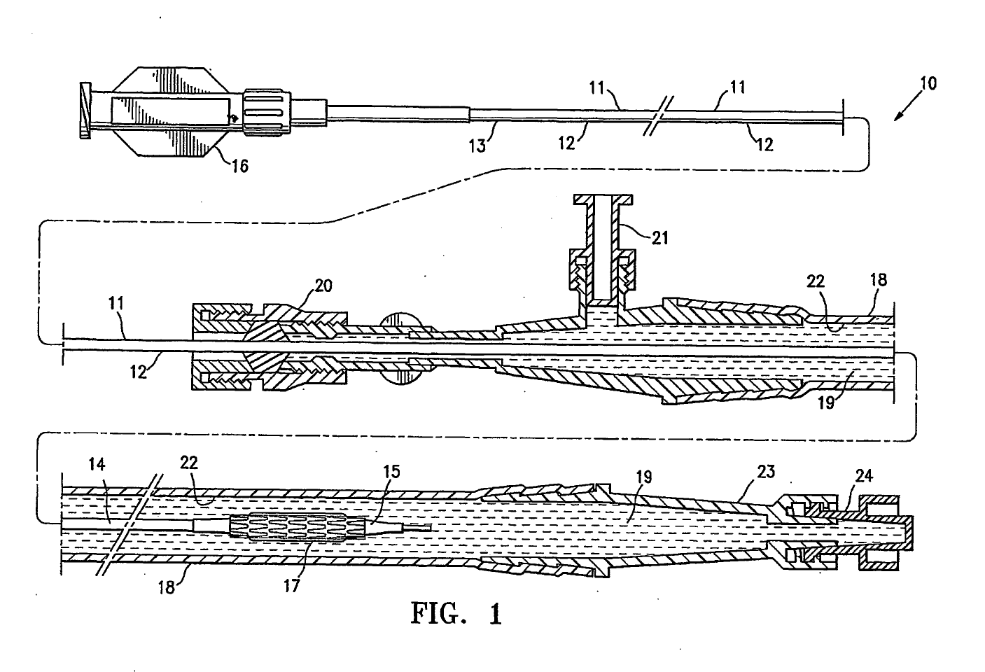

FIG. 1 illustrates a protected stent delivery system (also, a stent delivery device) 10 embodying features of the invention. The system 10 includes a delivery catheter 11 which has an elongated shaft 12 with a relatively stiff proximal shaft section 13, a flexible distal shaft section 14, a stent delivery member being an inflatable balloon 15 on the distal section and an adapter 16 mounted on the proximal end of the elongated shaft. An expandable stent 17 comprising a bovine pericardium jacket (see U.S. Pat. No. 6,468,300) is mounted on the working section of the balloon 15. A container being a protective sheath 18 is disposed about the distal shaft section 14. The protective sheath 18 is filled with protective fluid 19 (e.g., a glutaraldehyde solution, a saline solution) which minimizes detrimental affects to the stent 18 (e.g., to shield and protect stent 18, to prevent degradation of the jacket) and allows the stent to be used by the physician upon removal from the package (e.g., by maintaining the jacket of jacketed stent 18 in a condition that allows stent 18 to be used immediately upon removal from sheath 18) and possible rinsing the distal shaft section and mounted balloon before inserting into the patient.

The proximal end of the sheath 18 has a port being valve 20 configured to sealingly engage the exterior of catheter shaft 12. A side port 21 is also provided on the proximal end of the sheath 18 to allow for the introduction of fluid into and the withdrawal of fluids from the interior 22 of the sheath 18 to add and remove protective fluid and rinse the distal shaft section with an appropriate solution prior to use. The distal end 23 of the protective sheath 18 is provided with an accessing port 24 which either alone or in conjunction with the side port 21 at the proximal end of the sheath 17 allows for the introduction of fluids into and the withdrawal of fluids from the interior of the sheath 18. Ports 20 and 21 allow adding fluid for sterilizing, preserving and preparing for use stent 18 and components thereof. Ports 20 and 21 also allow rinsing or washing of stent 18 and components thereof.

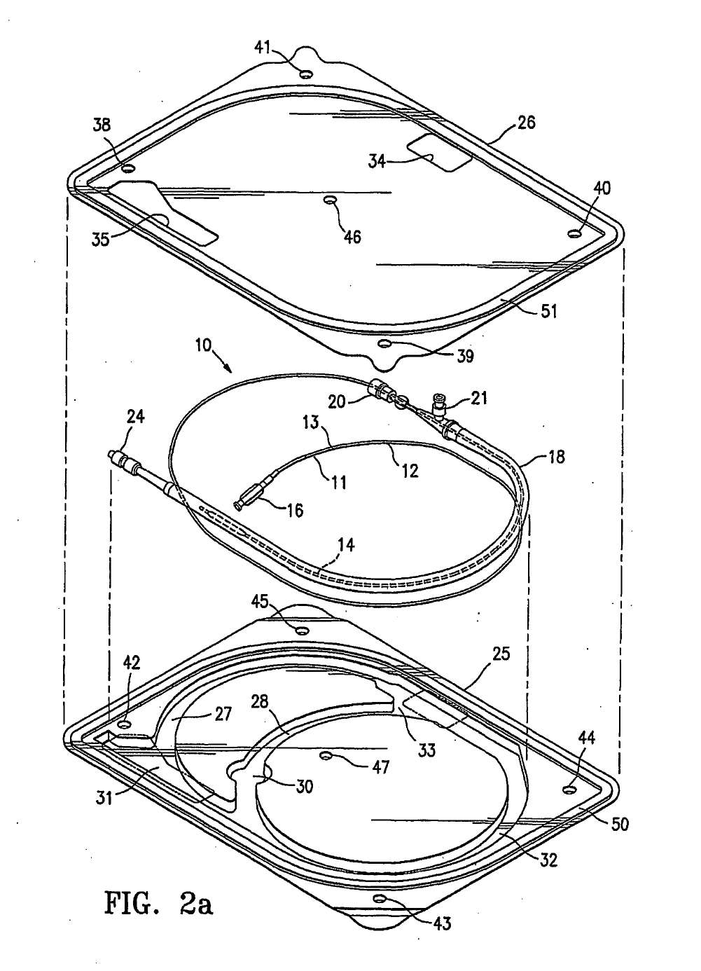

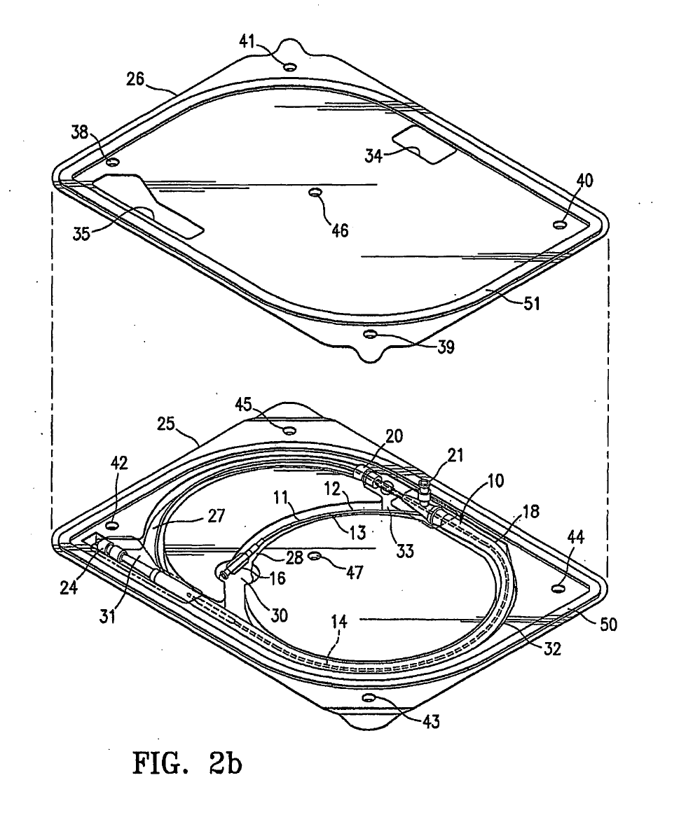

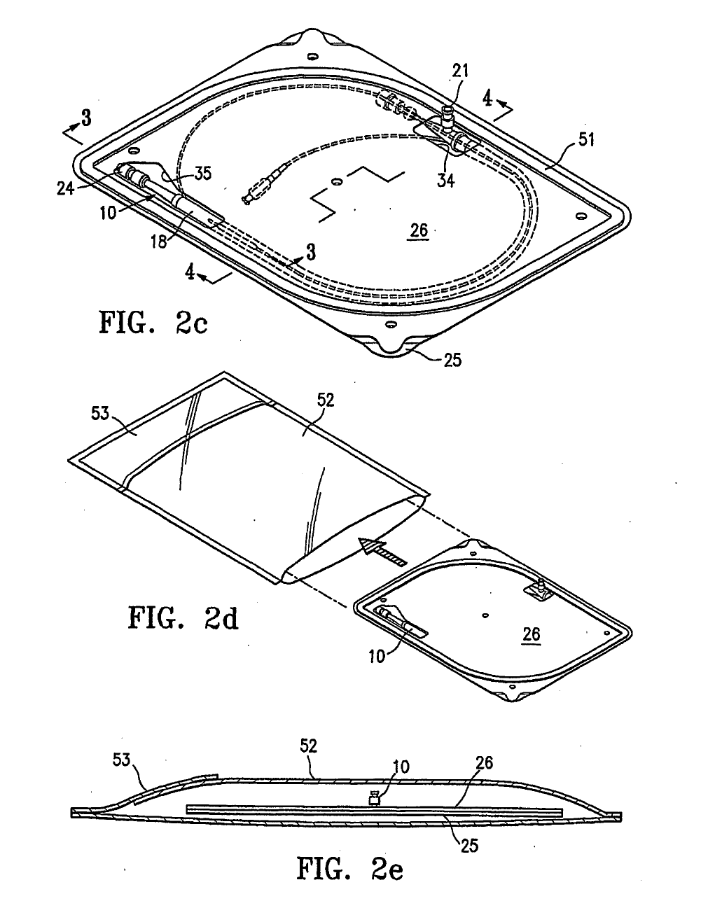

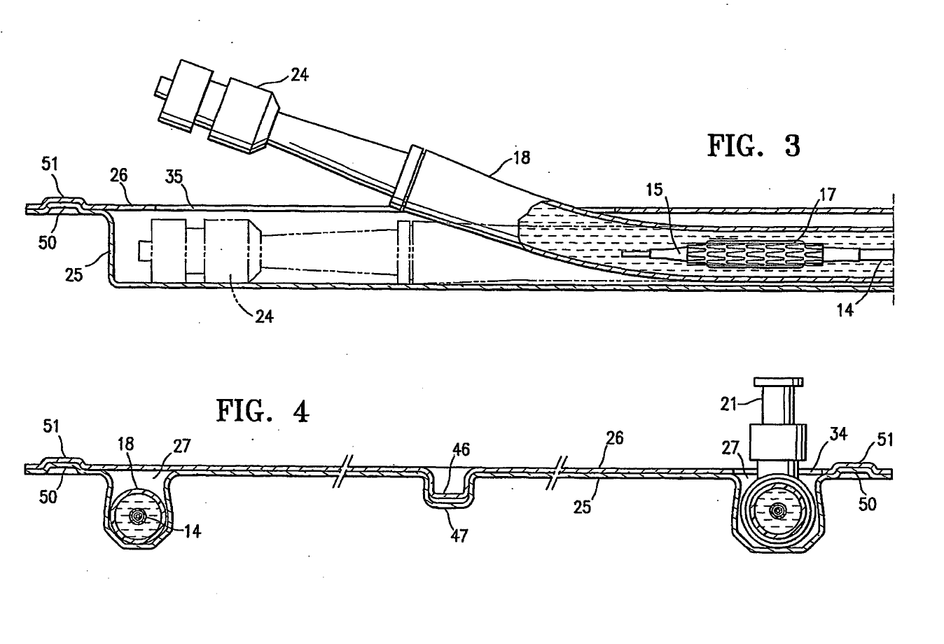

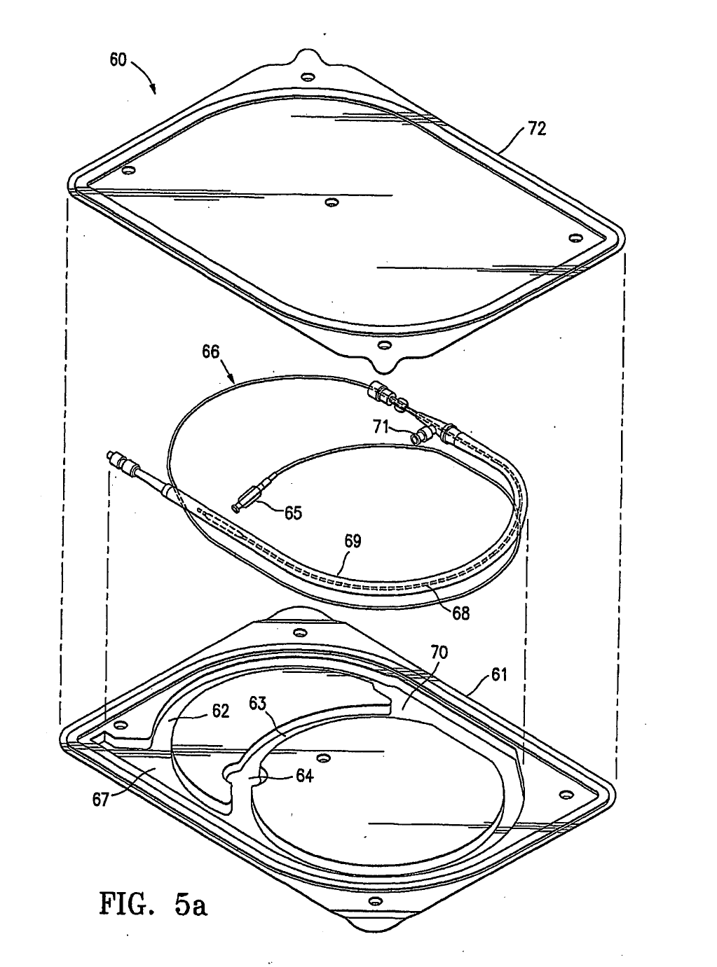

FIGS. 2a-2c illustrate an exploded view of a tray 25 and a tray cover 26 (together comprising a package including an interior volume of the present invention) suitable for the protected stent delivery system 10 with protective sheath 18 on the distal section 14. The upper surface of the tray 25 is provided with an oval recess 27 and an arcuate cross-over recess 28 configured to receive the stent delivery system 10. A well 30 is provided to receive the adaptor 16 at the proximal end of catheter shaft 12 and has larger dimensions that the crossover recess 28 to allow operating personnel to easily pick up the system 10 by the adapter 16. A run-out well 31 is provided to receive the distal end of the protective sheath 18. The sheath 18 extends from the run-out well 31 in the oval recess 27 around the curved end 32 thereof to the upper junction 33 between oval recess 27 and the cross-over recess 28. The tray cover 26 has a first opening 34 to allow the side port 21 (as shown in FIGS. 2c and 4) to extend therethrough. Similarly, the tray cover 26 has a second opening 35 to allow the distal end of the sheath 18 to be pulled through the opening to allow access to the accessing port 24 as shown in FIG. 3. This construction allows for the addition to or the withdrawal of fluids from the interior of the protective sheath 18, while the protected stent delivery system 10 remains protected in the covered tray. The corners of the tray cover 26 have projections 38-41 which fit into the corresponding recesses 42-45 at the corners of the tray 25 to provide a snap fit between the tray 25 and the cover 26. A centrally located projection 46 is provided in the cover 26 and a matching centrally location recess 47 is provided in the tray 25. A sealing ridge 50 is provided around the periphery of the upper surface of tray 25 and a matching sealing recess 51 is provided around the periphery of cover 26. As shown more clearly in FIG. 4 the recess 27 (and also recess 28 which is not shown) is provided with upper slightly overhanging lips to hold the stent delivery system in the recess during handling, transporting and storage while allowing its easy removal.

With protected stent delivery system disposed on the tray 25 and cover 26 in place, the covered tray may then be placed within a sealable envelope or pouch 52 as shown in FIG. 2d. Once the covered tray with stent delivery system 10 is placed within the interior of pouch 52, the lower edges of pouch 52 are sealed by heat or adhesive after the covered tray is inserted into the pouch. The securing flap 53 is presealed to the exterior of the pouch as shown in FIG. 2e and is configured to be used by the physician to open the pouch. At least part of the pouch 52 is formed of a permeable material such as Tyvek® which is permeable to a sterilizing gas such as ethylene oxide.

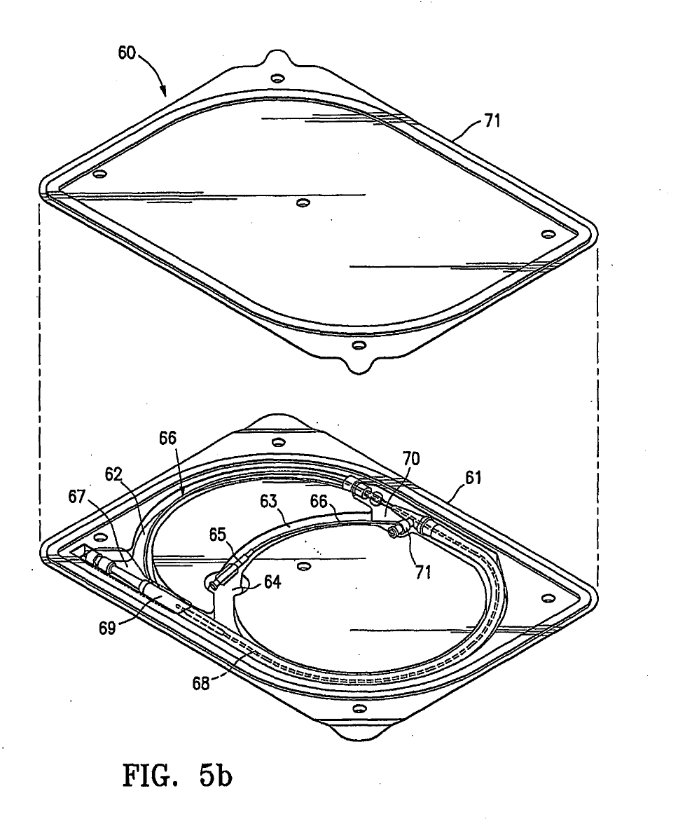

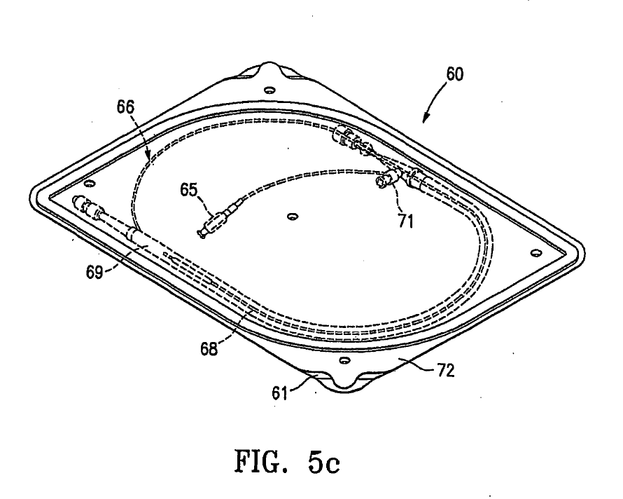

FIGS. 5a-5c illustrate an alternative sterilizable package system 60 (comprising a package including an interior volume of the present invention). The package system 60 has a tray 61 as described in the previous embodiment with an oval recess 62, an arcuate cross-over recess 63, circular well 64 configured to receive the adapter 65 from the delivery catheter 66 and run-out well 67 for the distal shaft section 68 with protective sheath 69. The tray 61 has an additional recess 70 configured to receive the side port 71 on the proximal portion of the sheath 69. The tray cover 72 is essentially the same as the tray cover described in the previously discussed embodiment except that it has no openings. This allows the tray and cover to be sealed and then sterilized as in the previous embodiment without the need for a pouch or other additional container. The tray cover may be formed at least in part of permeable materials.

Not depicted is an additional sterilizable package system of the present invention that comprises a tray 61 as depicted in FIGS. 5a-5c configured to hold a protected stent delivery system 10 and a tray cover that is substantially a sheet of Tyvek® that is attached to and sealed to tray 61 in the usual way (see, for example, the October 1999 Volume 8 Issue 3 version of the Medical Packaging Newsletter from Dupont).

Not depicted is an additional sterilizable package system of the present invention that comprises a tray 61 as depicted in FIGS. 5a-5c configured to hold a protected stent delivery system 10 that is placed inside a sealable pouch (or the like) made of a material such as Tyvek®, where tray 61 and the pouch comprise a package of the present invention.

Details of the stent and a stent jacket are described in U.S. Pat. No. 6,605,107, and a suitable stent delivery catheter is described in co-pending U.S. patent application Ser. No. 10/735,548 and PCT Patent Application No. IL 2004/001121 of the present inventor

The stent delivery catheter may be provided in either a rapid exchange design which has a proximal guide wire port a short distance, e.g. 5 to about 50, preferably about 10 to about 40 cm, from the distal end of the catheter or an over-the-wire design as described above in which the guide wire lumen extends the length of the catheter and is in fluid communication with the port in the proximal end of the catheter.

The catheter components may be formed of conventional materials used in angioplasty and stent delivery catheters and may be formed in a conventional manner. The elongated catheter shaft will generally have the dimensions of conventional dilatation or stent delivery catheters. The length of the catheter, measured from the distal end of the adapter 16 to the distal end of the elongated catheter shaft 11 may be about 90 cm to about 150 cm, and typically, it is about 137 cm.

The tubular protective sheath may be formed of suitable polymeric materials such as Tygon® or C-Flex® tubing and is configured to accommodate the distal extremity of the delivery catheter and a mounted stent.

The gas permeable portion of the container will typically be composed of a material which is permeable to the sterilizing gas and impermeable to liquids. Suitable gas permeable materials include high-density polyethylene (HDPE), typically a spun HDPE or other spun olefin. Commercially available materials such as Tyvek® (2FS, 1059B and 1073B) are specifically designed for packaging medical devices. Typically, the available materials are coated partially or totally with an adhesive for the purpose of sealing the material to itself or to adjacent materials.

The permeability required for the sterilizing gas to permeate into the interior of the container and contact the one or more articles therein, can vary depending upon the pressure differential, the temperature and the time available for permeation of the sterilizing gas into the interior. Preferably, the permeability is such that gases produced in the sterilization process (e.g., water produced during ethylene oxide sterilization) are allowed to escape.

The container may have both a gas permeable portion, as described above, and a gas impermeable portion, typically formed from a metal or polymer film which is impermeable to gases. Exemplary suitable material for the gas impermeable portion include metal foils such as aluminum, polymer films such as polyethylene, polyester, polyester (PET) modified low density polyethylene (LDPE) laminated film, or a laminate (e.g., metallized PET).

The permeable portion or portions of the container may be incorporated with suitable oxygen scavenger materials or may be positioned adjacent to a partial or complete film of suitable oxygen scavengers. Not only oxygen is removed from the package, oxygen gas trying to permeate through the permeable portion will be limited from entering the package.

Oxygen scavengers can be placed in fluid communication with the interior of the package, such as within a permeable packet that is placed within the interior of the container or within the interior of an enclosure surrounding the container. Oxygen scavengers may be embedded in the material of the container or in a pouch which receives the container.

While particular forms of the invention have been illustrated and described, it will be apparent that various modifications and improvements can be made to the invention. For example, the stent may be coated or provided with a jacket having one or more therapeutic or diagnostic agent incorporated therein. Unless described otherwise, conventional materials and methods of construction may be used to make the catheters and stents. Although individual features of embodiments of the invention may be shown in some drawings and not in others, those skilled in the art will recognize that individual features of one embodiment of the invention can be combined with any or all the features of another embodiment. Accordingly, it is not intended that the invention be limited to the specific embodiments illustrated. It is therefore intended that this invention to be defined by the scope of the appended claims as broadly as the prior art will permit.

Terms such a “element”, “member”, “device”, “section”, “portion”, “step”, “means” and words of similar import when used herein shall not be construed as invoking the provisions of 35 U.S.C. §112(6} unless the following claims expressly use the term “means” followed by a particular function without specific structure or the term “step” followed by a particular function without specific action.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, the present invention is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A protected stent delivery system for a jacketed or coated stent, comprising:
a. a delivery catheter which has an elongated shaft, a distal extremity on the elongated shaft and a stent expanding member on the distal extremity of the catheter;
b. a jacketed or coated stent mounted on the stent expanding member; and
c. a sealable container disposed about the distal extremity of the catheter which has an interior configured to receive the expandable member and the stent mounted thereon and which is configured to maintain a protective fluid within the interior of the container.

2. The delivery system of claim 1 wherein the sealable container is a sheath which has an inner lumen.

3. The delivery system of claim 2 wherein the sheath has a proximal end and a hemostatic closure at the proximal end.

4. The delivery system of claim 3 wherein the sheath has a distal end and a first accessing valve at the distal end.

5. (canceled)

6. The delivery system of claim 3 wherein the hemostatic closure includes a second accessing valve which provides access to the inner lumen of the sheath.

7. The delivery system of claim 3 wherein the hemostatic closure is configured to sealingly engage the elongated shaft of the catheter proximal to the expandable member.

8. (canceled)

9. The delivery system of claim 1 wherein the interior of the sealable container is at least partially filled with protective fluid.

10. The delivery system of claim 1 wherein the stent has a jacket formed at least in part of heterologous tissue.

11. (canceled)

12. The delivery catheter of claim 10 wherein a therapeutic or diagnostic agent has been incorporated into the heterologous tissue.

13. (canceled)

14. The delivery catheter of claim 1 wherein the stent has a coating into which a therapeutic or diagnostic agent has been incorporated.

15-16. (canceled)

17. A sterilized package containing a stent mounted on an inflatable member of a delivery catheter, comprising:
a. a support tray configured to receive an elongated delivery catheter with a proximal end and a distal end, an inflatable member proximally spaced from the distal end, an expandable stent mounted on the inflatable member and a sheath which has an inner lumen configured to receive the inflatable member of the delivery catheter and the stent mounted thereon and which is configured to maintain a protective fluid within the inner lumen in contact with the stent; and
b. a tray covering which defines in part a package interior and which is configured to retain sterile conditions within the package interior.

18. The package of claim 17 wherein at least part of the tray or tray covering is formed of material which is permeable to a sterilizing gas.

19. The package of claim 17 wherein the sheath is configured to sealingly receive the inflatable member and stent mounted thereon within the inner lumen of the tubular member.

20. The package of claim 17 wherein the sheath is disposed within the interior of the package.

21. The package of claim 20 wherein the sheath has at least one sealable port which facilitates introduction or withdrawal of fluid into the inner lumen of the tubular member.

22. (canceled)

23. The package of claim 21 wherein the sheath has at least one port accessible from an exterior location of the package to allow fluid introduction or withdrawal from outside the package without interfering with the sterile conditions within the interior of the package.

24-28. (canceled)

29. A sterilized package, comprising:
a. a sealed container which has an inner chamber defined at least in part by a wall, which has at least a portion permeable to sterilizing gas and which has at least one sterilized article within the inner chamber; and
b. a sealed sheath which is disposed about at least a portion of the sterilized article and which has a protective fluid within an interior thereof.

30. The sterilized package of claim 29 wherein the at least one article is a stent.

31. (canceled)

32. The sterilized package of claim 30 wherein the stent is a drug eluting stent.

33-35. (canceled)

36. The sterilized package of claim 30 wherein the stent is mounted on a balloon of a delivery catheter.

37. (canceled)

38. The sterilized package of claim 29 wherein the container includes a tray and a cover.

39. The sterilized package of claim 38 wherein the tray and a tray cover are disposed within a pouch.

40. The-sterilized package of claim 39 wherein the pouch is formed at least in part of a material which is permeable to sterilizing gas.

41. A method of packaging and sterilizing a medical device, comprising:
a. providing a container which has an inner chamber defined at least in part by a wall and which has at least a wall portion permeable to sterilizing gas;
b. sterilizing at least a portion of a medical device;
c. surrounding at least a sterilized portion of the medical device with a sealable sheath having an interior configured to receive a fluid to protect the sterilized portion of the medical device disposed therein;
d. disposing the at least partially sterilized medical device and sheath within the inner chamber of the container; and
e. contacting an exterior part of the permeable portion of the container with sterilizing gas under conditions which cause the sterilizing gas to penetrate the permeable portion to sterilize at least a portion of the medical device within the inner chamber.

42. The method of claim 41 wherein the medical device is a delivery catheter with an inflatable balloon and a drug eluting stent mounted on the balloon.

43. (canceled)

44. A sterilized packaging system for a medical device or component thereof, comprising:
a. a support tray configured to receive a medical device or component, a sheath which has an interior which retains at least part of the medical device or component and which is configured to maintain a protective fluid within the interior in contact with the device or component; and
b. a tray covering which is sealingly secured about the margin thereof to the tray, which defines in part a package interior containing the medical device or component and the sheath and which is configured to retain sterile conditions within the package interior; and
c. at least part of the tray or tray covering formed of a material which is permeable to sterilizing gas but is impermeable to pathogens.

45. A stent delivery device for a stent, comprising:
a. an elongated shaft that is a component of a stent delivery catheter;
b. a stent delivery member on a distal extremity of said elongated shaft;
c. a stent mounted on said stent delivery member; and
d. a container configured to maintain a fluid within an interior volume, holding said stent delivery member and said stent within said interior volume.

46-48. (canceled)

49. The stent delivery device of claim 45, wherein at least part of said container sealingly engages said elongated shaft passing through said part of said container.

50. The stent delivery device of claim 45, further comprising at least one port configured to allow fluid communication to said interior volume.

51-55. (canceled)

56. The stent delivery device of claim 45 wherein said interior volume of said container is at least partially filled with a fluid.

57. The stent delivery device of claim 56, wherein said fluid comprises an active pharmaceutical ingredient.

58. The stent delivery device of claim 45, wherein said container is opaque to sterilizing radiation.

59. A packaged stent, comprising:
a. a package including an interior volume configured to receive a stent delivery device of claim 45 and to maintain sterility of said interior volume; and
b. a stent delivery device of claim 45 packaged within said interior volume of said package.

60-64. (canceled)

65. The packaged stent of claim 59, wherein at least part of said package is substantially transparent to sterilizing radiation.

66. The packaged stent of claim 59, wherein at least part of said package is permeable to a sterilizing gas.

67. A packaged medical device, comprising:
a. a package including an interior volume configured to receive a medical device and to maintain sterility of said interior volume; and
b. a medical device packaged within said interior volume of said package. wherein a part of said medical device is held within a container configured to maintain a fluid within an interior volume of said container.

68-78. (canceled)

79. A method for packaging a medical device, comprising:
a. providing a medical device comprising a first part and a second part;
b. placing said first part within a container configured to maintain fluid within an interior volume of said container and sealing said container;
c. packaging said medical device in an interior volume of a package configured to maintain sterility of said interior volume;
d. while said medical device is packed within said package, sterilizing at least part of said medical device.

80-88. (canceled)
